(19) United States
(12) Patent Application Publication
(10) Pub. No.: US 2008/0033438 A1
(43) Pub. Date: Feb. 7, 2008

(54) Cervical Saddle Plate

(76) Inventor: Roy Frizzell, Boise, ID (US)

Correspondence Address: DYKAS, SHAVER & NIPPER, LLP, P.O. BOX 877, BOISE, ID, 83701-0877, US

(21) Appl. No.: 11/834,448

(22) Filed: Aug. 6, 2007

Related U.S. Application Data
(60) Provisional application No. 60/821,507, filed on Aug. 4, 2006.

(51) Int. Cl. A61B 17/58; A61B 17/56

(52) U.S. Cl. 606/69; 606/104; 606/73

(57) ABSTRACT

Presented is a cervical saddle plate and a cervical saddle plate assembly for stabilization of vertebrae in surgical procedures on humans. The assembly includes an anterior cervical plate, which is held in position on the vertebrae by screws which pass through screw passages in the plate body. At least some of the screw passages include a recess well which provides inter-fitting engagement with a cervical saddle plate which may be attached in a subsequent operation. By providing for interlocking engagement of the anterior cervical plate and cervical saddle plate, additional vertebrae can be stabilized without the need to remove the previously installed anterior cervical plate.

### CROSS-REFERENCE TO RELATED APPLICATIONS

This application claims the priority date of the provisional application entitled CERVICAL SADDLE PLATE filed by Dr. Roy Tyler Frizzell on Aug. 4, 2006 with application Ser. No. 60/821,507.

### FIELD OF THE INVENTION

The invention relates generally to a device for use in surgery on human vertebrae, and more particularly to a device for stabilizing vertebrae by use of a plate attached to the vertebrae.

### BACKGROUND OF THE INVENTION

When the vertebrae or the disks in a person's neck (cervical spine) begin to have problems, the disks may rupture and deteriorate, and this can result in nerve compression manifested by paralysis, numbness, or pain. The vertebrae can also form arthritic spurs which can pinch the nerves. One procedure that is performed to address this problem is to remove the disk, fuse the vertebrae, and install what is called a cervical plate. The cervical plate is a metal plate, typically made of titanium, and is designed to be secured to the vertebrae above and below the fusion with screws. The screws are similar to wood screws in appearance. The plate (which acts as an internal “cast”) spans the gap between two adjacent vertebrae, and the screws go through holes in the plates and into the vertebrae. Each vertebra may receive one screw, or may receive two screws depending on the type of plate which is utilized. The plate can span the distance between two vertebrae, three vertebrae or more. Once the fusion bone and cervical plate is in place, the vertebrae are stabilized and the bony fusion occurs according to biological healing principles. This prevents the nerves between them from becoming “re-pinched”.

These plates are typically placed on the anterior side of the spine. The posterior aspect of the spine is the side facing a person's back, and the anterior spine is the aspect facing a person's front side. Thus, to place these cervical plates, the tissue in front of the vertebrae must be moved to one side to expose the vertebrae. This involves moving the larynx, the pharynx, the esophagus, carotid artery, several important nerves, and dissecting several muscles. Gaining access to the anterior vertebrae surface is a delicate process and results in surgical trauma to the anterior neck structures.

A problem that often develops following a cervical fusion and placement of a cervical plate is that the vertebrae adjacent (above and below) to the stabilized vertebrae may also deteriorate and require a similar stabilization. The estimated frequency of this occurrence may exceed 25% in 10 years following the initial fusion. When this occurs, the typical procedure is to redissect the anterior neck structures, expose the anterior cervical plate, and remove it. Next, the tissue over the newly deteriorated vertebrae must be exposed, the disk removed and a fusion bone placed. Then, a longer plate is installed which covers the original two vertebrae and one or more adjacent newly fused vertebrae. Removing the old plate is complicated by difficulty in exposing the old plate because surgical scarring commonly occurs. Installing the longer new plate exposes the patient to a great deal of trauma (bleeding, hoarseness, trouble swallowing, esophageal injury, loose metal fragments from drilling the old plate), and can be technically difficult for the surgeon.

### SUMMARY OF THE INVENTION

The invention is a cervical saddle plate which is configured to be attached to an anterior cervical plate (in a “saddle” fashion) and which allows the previously installed anterior cervical plate to remain in place when additional stabilization needs to be extended to adjacent vertebrae. The cervical saddle plate has a plate body with an overlap plate and a bone contact plate. The overlap plate of the plate body includes screw passages, and around the screw passages on the overlap plate includes convex bulge for overlapping engagement with corresponding screw holes of the underlying anterior cervical plate, and allows a screw to pass through both the overlap plate and the original cervical plate. The bone contact plate of the cervical saddle plate also has screw passages to allow attachment to underlying vertebrae. To install the cervical saddle plate of the invention, the screw(s) at the end of the previously installed anterior cervical plate are removed, and the cervical saddle plate is placed over the end of the original plate, so that the screw hole(s) overlap with the screw holes of the original cervical plate, and a replacement screw(s) can be screwed in place, securing the end of the original plate as well as the overlap plate of the cervical saddle plate. The bone contact plate of the cervical saddle plate is made to be coplanar with the original anterior cervical plate, and has one or more screw holes which are spaced for attachment to an adjacent vertebra. The cervical saddle plate can cover one or more adjacent vertebra. One saddle plate can also be overlapped with another saddle plate; so, conceivably, starting with one anterior cervical plate, additional saddle plates can be attached if a third surgical procedure is necessary.

The cervical saddle plates can include a locking mechanism which moves from an open position to a closed position, and would cover the exit paths of the screw heads and prevent the screw heads from working loose. The screws used to replace the original screws in the cervical plate may be longer than the original screws in order to secure sufficient purchase in the bone material. They can also be wider with more aggressive threads to ensure that they secure a positive grip in the bone. The screw holes in the overlap plate portion of the cervical saddle plate body can be formed in screw wells in the overlap plates, with recessed screw head sockets on the anterior or first side of the overlap plate, and with projecting screw sockets on the posterior or second side of the overlap plate, with the projections configured to fit inside the screw holes of the original cervical plate, and thus help to lock the two plates together. Thus, the cervical saddle plate provides stability to the adjacent vertebrae in terms of translational, vertical, horizontal and rotational stability.

This method for providing stability to adjacent vertebrae provides a biomechanically advantageous method of stabilization which would have significant advantages over the current “remove and re-plate” method described above. Other methods to fuse adjacent vertebrae, such as utilizing a “buttress” plate (a rectangular piece of metal which hangs over the new fusion fixed at only one end) or a Cloward Fusion (a dowel of fusion bone placed without metal fixation) are biomechanically inferior and have relatively low fusion rates.

A version of the cervical saddle plate of the invention can also be utilized in a situation in which a disk adjacent to an existing cervical plate is removed and replaced with an artificial disk (AD) instead of a fusion bone. This may be preferable in some cases since additional fusion is avoided. The cervical saddle plate of this version would have a plate overlap portion and a first support bracket portion (the stub plate) in the form of an L shaped projection, with the overlap plate connected to the support bracket portion by an artificial disc. For instance, a cervical saddle plate for AD can be attached to an existing anterior cervical plate, and in this instance two screws pass through the cervical saddle plate, and through the anterior cervical plate and secure the two to the underlying vertebrae. The cervical saddle plate for AD can comprise a saddle plate assembly which includes an L-shaped support bracket which is configured so that the disk surface inferior to vertebrae 3 is supported by the L-shaped projection as if on a shelf. The vertebrae 5 is held in a spaced apart position from vertebrae 3 by the L-shaped projection (extending into the disk space) on the adjacent spacing bracket (stub plate).

The purpose of the foregoing Abstract is to enable the public, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other features and advantages of the present invention will become readily apparent to those skilled in this art from the following detailed description describing preferred embodiments of the invention, simply by way of illustration of the best mode contemplated by carrying out my invention. As will be realized, the invention is capable of modification in various obvious respects all without departing from the invention. Accordingly, the drawings and description of the preferred embodiments are to be regarded as illustrative in nature, and not as restrictive in nature.

### BRIEF DESCRIPTION OF THE DRAWINGS

FIG. 1 is a perspective view of the first side of a cervical saddle plate of the invention.

FIG. 2 is a perspective view of the second side of the cervical saddle plate of the invention.

FIG. 3 is a perspective view of the locking plate of the invention.

FIG. 4 is a perspective view of an embodiment of the cervical saddle plate assembly of the invention.

FIG. 5 is a perspective view of an embodiment of the cervical saddle plate assembly of the invention.

FIG. 6 is a perspective view of an embodiment of the cervical saddle plate assembly of the invention.

FIG. 7 is perspective view of an embodiment of the cervical saddle plate assembly of the invention.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

Presented is a cervical saddle plate and a cervical saddle plate assembly for stabilization of vertebrae in surgical procedures on humans. The assembly includes an anterior cervical plate, which is held in position on the vertebrae by screws which pass through screw passages in the plate body. At least some of the screw passages include a recess well which provides inter-fitting engagement with a cervical saddle plate which may be attached in a subsequent operation. By providing for interlocking engagement of the anterior cervical plate and cervical saddle plate, additional vertebrae can be stabilized without the need to remove the previously installed anterior cervical plate.

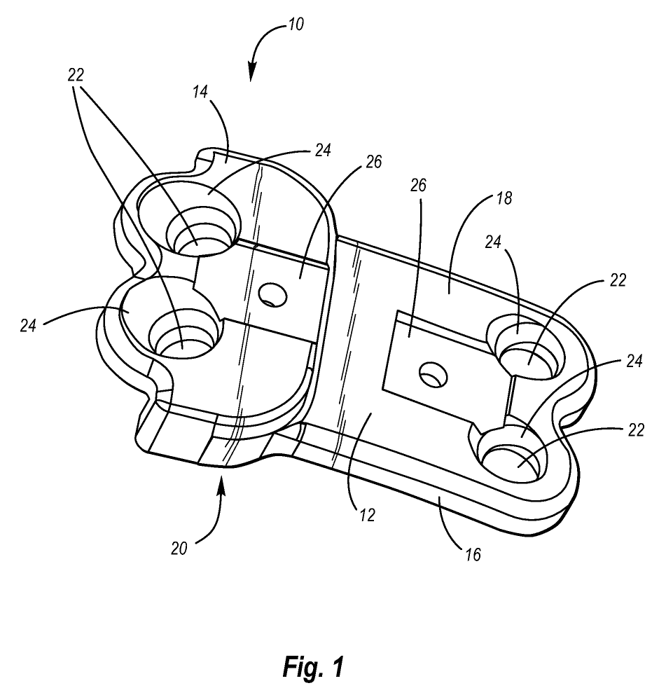

FIG. 1 shows a cervical saddle plate 10 of the invention. The cervical saddle plate 10 includes a plate body 12, with the plate body being made of an overlap plate 14 and a bone contact plate 16. The overlap plate 14 and the bone contact plate 16 form a bi-plainer plate body. The plate body 12 has a first side 18 and a second side 20, with the first side 18 being visible in FIG. 1.

Defined in the plate body 12 are four screw passages 22. The screw passages 22 are provided for passage of a screw (not shown), with a threaded shaft and a screw head, through the cervical saddle plate 10 and into underlying bone. The cervical saddle plate 10 shown in FIG. 1 with the screw passages 22 is configured so that the screw passages 22 in the overlap plate 14 are aligned so that screws can pass through them and through similar screw passages in a cervical plate mounted on a patient's vertebrae. In this way a cervical plate which has previously been installed on a patent's vertebrae, need not be removed to extend the stabilization to adjacent vertebrae. The cervical saddle plate 10 of FIG. 1 has two screw passages 22 in the overlap plate and two screw passages 22 in the bone contact plate 16.

Associated with each of the screw passages 22 is a concave screw head well 24. The concave screw head well provides a recess for the head of the screw so that when it is fully installed, the head of the screw does not protrude above the surface of the cervical saddle plate 10.

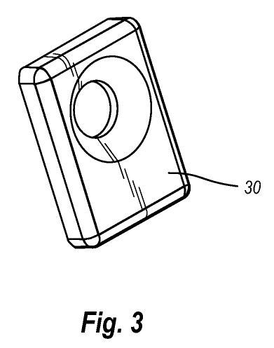

Also shown in FIG. 1 are a first and second screw lock recess 26 and 28. These recesses provide a mounting region for a screw lock 30 which is shown in FIG. 3. Screw lock 30 is secured in place in the first and second screw lock recess 26 and 28 in order to prevent a screw from backing out of its engagement with the underlying bone.

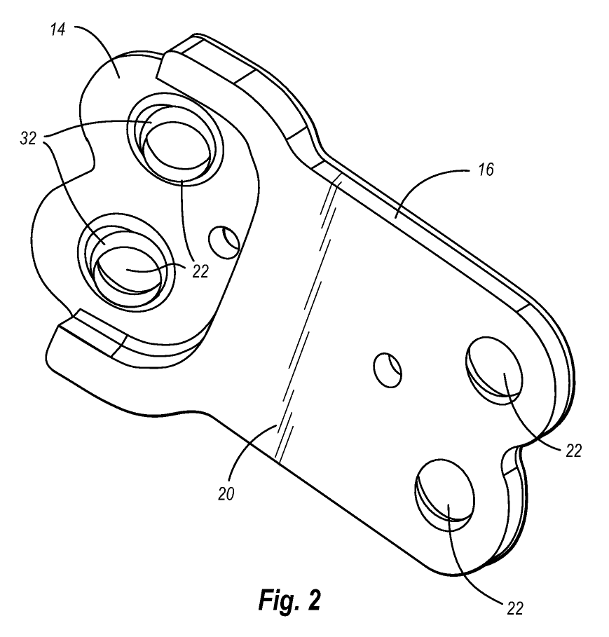

FIG. 2 is a view of the same embodiment shown in FIG. 1, and shows the second side 20 of the cervical saddle plate 10, and shows the screw passages 22 in the plate body 12. Around the screw passages 22 in the overlap plate 14 is a convex screw head bulge 32. The convex screw head bulge 32 surrounds the screw passages 22 as well as the screw when it is inserted through those pages. The convex screw head bulge 32 is configured to lock in place in the concave screw head wells 24 of an underlying cervical plate 50. In this way the cervical plate 50 which is shown in other figures and the cervical saddle plate 10 interlock to form a rigid and secure vertebrae stabilization device, which can be added to a previously mounted cervical plate, to extend stabilization to one or more adjacent vertebras.

FIG. 3 shows a screw lock 30, which would be screwed in place in the first and second screw head recess 26 and 28 which are shown in FIG. 1. When secured in place, the screw lock 30 would overlap the top edge of a screw head (not shown) and prevent the screw head from backing out of its engagement with the vertebrae.

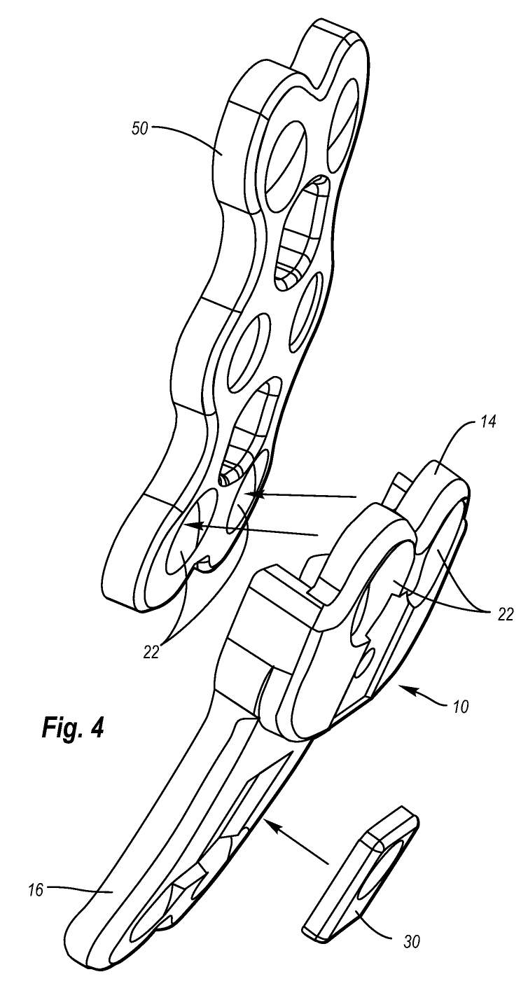

FIG. 4 shows a cervical plate 50, a cervical saddle plate 10, with the cervical saddle plate 10 having an overlap plate 14, a bone contact plate 16, and a screw lock 30. Together these pieces form a cervical saddle plate assembly. Screws would pass through the screw passages 22 of the cervical saddle plate 10 and into corresponding screw passages 22 on the cervical plate 50. In this way if the cervical plate 50 has been mounted to vertebrae in a previous operation, the cervical saddle plate 10 could be added it in a subsequent operation so that the cervical plate 50 would not have to be removed and replaced with a longer stabilization device.

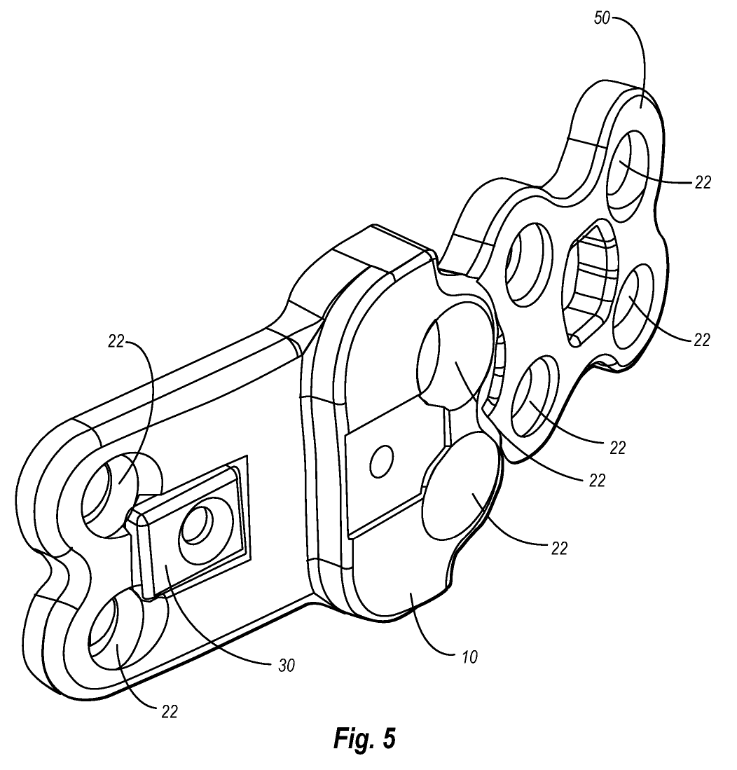

FIG. 5 is a view of the same components, showing them in the assembled position, with the cervical saddle plate 10 engaged with the cervical plate 50, and with one screw lock 30 in place to hold the screws in the screw passages 22.

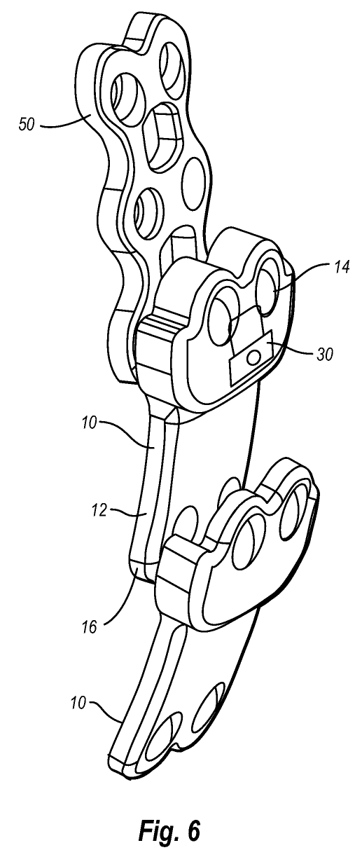

FIG. 6 shows another arrangement of the same pieces from above. FIG. 6 shows a cervical plate 50 to which is attached a cervical saddle plate 10 having a second saddle plate 10 attached to it. In this way, vertebra stabilization can be extended as needed without removing prior appliances, or to tailor a longer stabilization appliance for lengths of specific vertebrae.

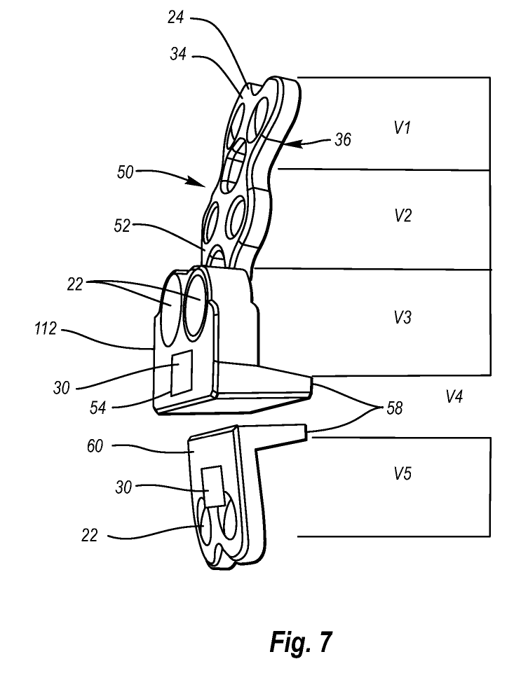

FIG. 7 shows another preferred embodiment of the invention which includes a cervical plate 50 in which the cervical plate has a first side 34 and a second side 36. The second side 36 is configured for placement toward the vertebrae, the cervical plate body 52 includes a plurality of screw passages 22. At least two of the screw passages 22 include within their structure a concave screw head well 24 on the first side of the cervical saddle plate body 52. These concave screw head wells 24 are configured for a corresponding engagement with a convex screw head bulge 32 on the second side of a cervical saddle plate 10 as shown in FIGS. 4, 5 and 6. FIG. 7 includes a cervical saddle plate body 112 with a first cervical plate bracket 54. A stub plate 60 is configured to be used with the anterior cervical plate and the cervical saddle plate of FIG. 7, to provide stabilization and support in the region of missing vertebrae 4. The stub plate 60 and the cervical saddle plate body 112 each include an L-shaped plate body 58 configured to extend between two vertebrae, and to hold two vertebrae in a spaced apart position to maintain an opening where a vertebrae v4 has been removed between the two vertebrae. Shown in FIG. 7 are vertebras 1, 2, 3 and 5, with vertebrae 4 having been removed. The L-shaped plate body 58 of the first cervical plate bracket and the second cervical plate bracket 54 and 56 extend into the space previously occupied by vertebrae 4, and maintain the gap between vertebrae 3 and vertebrae 5. As in the cervical saddle plate of FIG. 1, these cervical plates form a bi-planar plate body with the planes being at approximately 90 degrees to each other. In FIG. 1, the cervical saddle plate 10 has a bi-planar plate body, with the planes being approximately parallel and offset with each other. The device in FIG. 1 has similar structures such as screw passages 22.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

1. A cervical saddle plate for attachment to one or more human vertebrae and for corresponding engagement with a cervical plate comprising:
a plate body, with said plate body comprising an overlap plate, and a bone contact plate, and a first side and a second side, with said overlap plate and said bone contact plate forming a generally bi-planar plate body in cross section, with said first side configured for placement away from said vertebrae, and said second side configured for placement toward said vertebrae, with said second side of said overlap plate configured for corresponding engagement with said cervical plate, and with said bone contact plate configured for attachment to said human vertebrae in a position coplanar with said cervical plate; and
a means of attachment to said vertebrae.

2. The cervical saddle plate of claim 1 in which said cervical saddle plate defines one or more screw passages in said cervical saddle plate for passage of one or more screws, each with a screw head and a threaded shaft, for attachment to said vertebrae.

3. The cervical saddle plate of claim 2 in which said cervical saddle plate defines one or more screw passages in said overlap plate which are configured for corresponding engagement with one or more screw passages in said cervical plate, so that said screw passages in said overlap plate may be placed in corresponding engagement over screw passages in said cervical plate, and one screw may be used to secure both said cervical plate and said cervical saddle plate at the selected screw passage, with said bone contact plate further comprising one or more screw passages, for passage of one or more screws into said vertebrae.

4. The cervical saddle plate of claim 3 in which said cervical saddle plate further comprises two screw passages in said overlap plate and two screw passages in said bone contact plate.

5. The cervical saddle plate of claim 4 in which said cervical saddle plate defines a concave screw head well on a first side of said overlap plate for each screw head, and further comprising a convex screw head bulge for each screw head on a second side of said overlap plate, with said bulges configured for engagement with corresponding wells of said cervical plate.

6. The cervical saddle plate of claim 5 which further comprises a screw lock adjacent each screw head well, with each screw lock having an open and a closed position, with said closed position locking said screw head in place and preventing backing out of said threads.

7. The cervical saddle plate of claim 6 in which said screw lock comprises a lock plate configured to at least partially overlap one or more of said screw heads.

8. The cervical saddle plate of claim 7 in which said lock plate partially overlaps a portion of two screw heads.

9. A cervical plate assembly for attachment to human vertebrae, with said cervical plate assembly comprising:
a cervical plate body with a first side and a second side, with said first side configured to face away from said vertebrae, and with said second side configured for placement toward said vertebrae, with each said cervical plate body defining a plurality of screw passages for passage of a screw into said vertebrae, with at least two screw passage further comprising a concave screw head well on a first side of said cervical plate body, configured for corresponding engagement with a corresponding convex screw head bulge on a second side of a cervical saddle plate;
a cervical saddle plate, comprised of an overlap plate, and a bone contact plate, and a first side and a second side, with said overlap plate and said bone contact plate forming a generally bi-planar plate body in cross section, with said first side configured for placement away from said vertebrae, and said second side configured for placement toward said vertebrae, with said second side of said overlap plate configured for corresponding engagement with said cervical plate, and with said bone contact plate configured for attachment to said human vertebrae in a position coplanar with said cervical plate, with said cervical saddle plate defining a plurality of screw passages for passage of a screw into said vertebrae, with said screw passages on said overlap plate further comprising for each screw passage, a convex screw head bulge positioned on a second side of said overlap plate, with said bulges configured for engagement with corresponding screw head wells of said cervical plate.

10. The cervical plate assembly of claim 7 which further comprises for each screw passage a concave screw head well on a first side of said overlap plate for flush engagement of each screw head.

11. The cervical plate assembly of claim 9 which further comprises a screw lock adjacent each screw head well, with each screw lock having an open and a closed position, with said closed position locking said screw head in place and preventing backing out of said threads.

12. The cervical plate assembly of claim 11 in which said screw lock comprises a lock plate configured to at least partially overlap one or more of said screw heads.

13. The cervical plate assembly of claim 12 in which said lock plate partially overlaps a portion of two screw heads.

14. A cervical plate assembly for attachment to human vertebrae, with said cervical plate assembly comprising:
a cervical plate body with a first side and a second side, with said first side configured to face away from said vertebrae, and with said second side configured for placement toward said vertebrae, with said cervical plate body defining a plurality of screw passages for passage of a screw into said vertebrae, with each screw passage further comprising a concave screw head well on a first side of said cervical plate body, configured for corresponding engagement with a corresponding convex screw head bulge on a second side of said cervical plate body;
a cervical saddle plate body, with said saddle plate body comprising an overlap plate and a bracket portion, with said overlap plate and said bracket portion forming a generally bi-planar and L shaped plate body, with said cervical saddle plate defining a plurality of screw passages, for passage of a screw into said vertebrae, with said screw passages on said overlap plate further comprising for each screw passage, a convex screw head bulge positioned on a second side of said overlap plate, with said bulges configured for engagement with corresponding screw head wells of said cervical plate with said bulges of said overlap plate configured for corresponding engagement with said wells of said cervical plate, and with said bracket portion configured for contact with an inferior surface of a first vertebrae in a position generally normal to said cervical plate, for spacing of said vertebrae from an adjacent second vertebrae.

15. The cervical plate assembly of claim 14 which further comprises for each screw passage a concave screw head well on a first side of said overlap plate for flush engagement of each screw head.

16. The cervical plate assembly of claim 15 which further comprises a screw lock adjacent each screw head well, with each screw lock having an open and a closed position, with said closed position locking said screw head in place and preventing backing out of said threads.

17. The cervical plate assembly of claim 16 in which said screw lock comprises a lock plate configured to at least partially overlap one of more of said screw heads.

18. The cervical saddle plate of claim 17 in which said lock plate partially overlaps a portion of two screw heads.

19. A cervical saddle plate assembly for attachment to a human vertebrae and for corresponding engagement with a cervical plate comprising;
a plate body, with said plate body comprising an overlap plate and a bracket portion, with said overlap plate and said first support bracket portion forming an L shaped plate body, with said overlap plate configured for corresponding engagement with said cervical plate, and with said first support bracket portion configured for contact with an inferior surface of said human vertebrae in a position generally normal to said cervical plate; and
a spacing bracket with a bracket body, with said bracket body comprising a bone contact plate and a second support bracket portion, with said bone contact plate and said second support bracket portion forming an L shaped plate body, with said bone contact plate configured for attachment to a human vertebrae, and with said second support bracket portion configured for contact with a superior side of said human vertebrae in a position generally normal to said cervical plate, with said first and second support bracket configured for maintaining a space between the two brackets to which they are attached.
